(19) United States
(12) Patent Application Publication
(10) Pub. No.: US 2011/0008634 A1
(43) Pub. Date: Jan. 13, 2011

(54) RUST INHIBITOR AND SURFACE TREATMENT METAL MATERIAL

(75) Inventors: Tatsuya Hase, Yokkaichi-shi (JP); Makoto Mizoguchi, Kasuga-shi (JP)

Correspondence Address: OLIFF & BERRIDGE, PLC, P.O. BOX 320850, ALEXANDRIA, VA, 22320-4850, US

(73) Assignees: AUTONETWORKS TECHNOLOGIES, LTD., YOKKAICHI-SHI (JP); SUMITOMO WIRING SYSTEMS, LTD., YOKKAICHI-SHI (JP); SUMITOMO ELECTRIC INDUSTRIES, LTD., OSAKA-SHI (JP); KYUSHU UNIVERSITY, FUKUOKA-SHI (JP)

(21) Appl. No.: 12/922,464

(22) PCT Filed: Jul. 2, 2009
(86) PCT No.: PCT/JP2009/062084

§ 371 Date: Sep. 14, 2010

(30) Foreign Application Priority Data
Aug. 11, 2008 (JP) JP2008-206523
Nov. 28, 2008 (JP) JP2008-303887

(51) Int. Cl. B32B 15/04; C07C 229/26; C07C 69/72; C07C 229/24; C07F 9/38; C07J 53/00; C07C 35/37

(52) U.S. Cl. 428/457; 560/169; 560/178; 560/252; 560/251; 558/198; 552/514; 568/818

(57) ABSTRACT

A rust inhibitor usable for metal surface coating includes, as an effective component, a compound containing a chelate group, and a long chain alkyl group and/or a cyclic alkyl group, which are bonded by an ester bond or other bonds. The compound is obtained by reacting a chelate ligand having the chelate group, such as aminocarbonic acid, acetoacetic acid, acetoacetic ester, and hydroxycarbonic acid, with a compound having the long chain alkyl group and/or the cyclic alkyl group, such as long chain (cyclic) alkyl carbonic acid and long chain (cyclic) alkyl alcohol,

### TECHNICAL FIELD

The present invention relates to a rust inhibitor and a surface treatment metal material, and more particularly, to a rust inhibitor that is suitable to be coated on metal surfaces of various metal materials in order to prevent generation of rust, and a surface treatment metal material using the same.

### BACKGROUND ART

In the related art, metal materials are used in various fields, and metal materials take on an important role in industry fields. However, because metal materials easily rust, it is required that metal materials be subjected to rust inhibition treatment in order to stably perform its role over a long period of time. Accordingly, with respect to various metal materials, various rust inhibiting methods according to the metal species have been proposed.

As the rust inhibiting methods for metal materials, for example, a method of performing plating on a metal surface and a method for painting a metal surface have been well known. The above methods are used to prevent affection of factors of rust, such as water or oxygen, and show a rust inhibiting effect by forming a coat on a metal surface and physically covering the metal surface. However, the plating or painting may be a large-scale process.

On the other hand, as a relatively simple method, a method for coating a rust inhibitor on a metal surface is known. For example, a method for coating VASELINE or grease on a metal surface is known. In addition, Patent Literature 1 discloses a method for coating a rust inhibitor on the surface of zinc-based plated steel or aluminum-based plated steel, and a method for forming a coat by a polymer chelating agent using a specific polyamino compound as an organic polymer resin matrix on the metal surface.

### CITATION LIST

### Patent Literature

PLT1: Japanese Laid-Open Patent Publication No. Hei 11-166151

### DISCLOSURE OF THE INVENTION

### Problem to be Solved by the Invention

However, in the known method for coating various kinds of Vaseline or grease on the metal surface, they may be easily volatilized or eluted by heat or a solvent. Consequently, a rust inhibiting effect may be reduced considerably.

In addition, the method for using various kinds of Vaseline or grease and the method for using the polymer chelating agent disclosed in Patent Literature 1 disclose that a rust inhibiting effect is obtained by coating the rust inhibitor on the metal surface to form a continuous coat on the metal surface and physically'covering the metal surface. Hence, the methods are significantly different from the present invention in terms of constitution and function.

It is an object of the present invention to provide a rust inhibitor that has an excellent adhering property to a metal surface and may show a stable rust inhibiting effect over a long period of time, and a surface treatment metal material using the same.

### Means to Solve the Problem

The present inventors have conducted extensive studies, the results in the finding are that if a compound that has a portion having a bonding property with respect to a metal surface and a portion having a property for preventing water or oxygen from permeating the metal surface simultaneously is used as an effective component, a rust inhibiting effect may be stably shown over a long period of time while an adhering property to the metal surface is excellent.

To achieve the objects and in accordance with the purpose of the present invention, a rust inhibitor according to a preferred embodiment of the present invention includes a compound that has a hydrophobic group and a chelate group in a molecular structure.

It is preferable that the hydrophobic group is one or a plurality of groups selected from the group consisting of a long chain alkyl group and a cyclic alkyl group.

It is preferable that the chelate group is derived from one or a plurality of chelate ligands selected from the group consisting of polyphosphate, aminocarbonic acid, 1,3-diketone, acetoacetic acid, acetoacetic ester, hydroxycarbonic acid, polyamine, aminoalcohol, aromatic heterocyclic bases, phenols, oximes, Schiff's base, tetrapyrroles, sulfur compound, synthesized macrocyclic compound, phosphonic acid, and hydroxyethylidenephosphonic acid.

It is preferable that the hydrophobic group and the chelate group are bonded by one or a plurality of bonds selected from the group consisting of an ester bond, an ether bond, a thioester bond, a thioether bond, and an amide bond.

It is preferable that the compound is a neutral compound.

It is preferable that the rust inhibitor is used for metal surface coating.

A surface treatment metal material according to another preferred embodiment is formed by coating the rust inhibitor described above on a surface of a metal material.

It is preferable that the metal material is made of one or a plurality of metals selected from the group consisting of aluminum, iron, copper, an aluminum alloy, an iron alloy, and a copper alloy.

### Effects of the Invention

The rust inhibitor according to the present invention includes the compound that has the hydrophobic group and the chelate group in the molecular structure. Therefore, the adhering property to a metal surface is improved by bonding the chelate group to the metal surface. In addition, since the hydrophobic group that is connected to the chelate group faces toward the outside of the metal surface, the hydrophobic group may provide a water repellent property to the metal surface. Therefore, permeation of water is prevented. Accordingly, a rust inhibiting effect may be stably shown over a long period of time while an adhering property to a metal surface is excellent.

At this time, if the hydrophobic group includes various kinds of groups, the hydrophobic group may provide a water repellent property to the metal surface. At this time, if the chelate group includes various kinds of groups, the chelate group may be bonded to the metal surface. At this time, the bonding of the hydrophobic group and the chelate group by the various kinds of bonds may make the synthesis easy and may be widely used.

Herein, if the compound is a neutral compound, corrosion or an effect on the human body may be prevented, so that even if the rust inhibitor is attached to a portion that is not included in an intended coated side, the compound is excellent in safety. In addition, if the compound is a neutral compound, the compound is not easily affected by the environment and excellent in safety

Meanwhile, in the surface treatment metal material according to the present invention, since the rust inhibitor is coated on the surface of the metal material, a rust inhibiting effect may be stably shown over a long period of time.

### BEST MODE FOR CARRYING OUT THE INVENTION

Hereinafter, preferred embodiments of the present invention will be described in detail. The rust inhibitor according to a preferred embodiment of the present invention includes a compound that has a hydrophobic group and a chelate group in a molecular structure as an effective component. The rust inhibitor according to the present invention, for example, may be appropriately used so as to be coated on a metal surface of a metal material. Examples of the metal material include wires, cables, connectors, and bodies in vehicles such as automobiles, high voltage power cables, electric and electronic device parts. In addition, examples of the metal species include aluminum, iron, copper, an aluminum alloy, an iron alloy, and a copper alloy.

In the rust inhibitor according to the present invention, the chelate group is a portion that is formed to bond to the rust inhibiting metal surface. Since the chelate group bonds to the metal surface, the rust inhibitor is not easily volatilized or eluted by heat or a solvent. Accordingly, the rust inhibiting effect may be stably shown over a long period of time. The change of the chelate group to chelate bond through bonding to the metal surface may be confirmed by, for example, attenuated total reflectance IR absorption method (ATR-IR) or microscopic IR and the like.

In the rust inhibitor according to the present invention, the hydrophobic group is disposed so as to protrude from the chelate group that is formed by bonding it to the metal surface to the outside. The hydrophobic group has the water repellent property on the chelate group that is formed by bonding to the metal surface in order to prevent water from permeating the metal surface. That is, the rust inhibiting effect is obtained by physically covering the metal surface, and also by preventing water from permeating the metal surface due to a water repellent effect of the hydrophobic group.

It is preferable that the hydrophobic group and the chelate group are bonded by bonds such as an ester bond, an ether bond, a thioester bond, a thioether bond, and an amide bond. Through these bonds, the bonding structure of the hydrophobic group and the chelate group may be easily synthesized by a condensation reaction.

The compound that has the hydrophobic group and the chelate group may be any one of acidic, alkali, and neutral compounds. Preferably, it is neutral. In the case of when the compound is a neutral compound, even if the rust inhibitor is attached to a portion that is not included in an intended coated side, corrosion is not easily caused in the portion to which the rust inhibitor is attached. In addition, in the case of when the rust inhibitor is attached to a skin of a human body, an effect to the human body such as roughness of the skin is insignificant. That is, it is excellent in safety. In addition, in the case of when the compound is neutral, the compound is not easily affected by the environment as compared to an acidic compound or alkali compound. Therefore, it is excellent in preservation stability.

Examples of the neutral compound includes a compound that does not have an acidic structure or a base structure in a molecular structure (in this case, the chelate group does not have an acidic structure or a base structure), and a compound that has an acidic structure and a base structure in a molecular structure but is neutral.

The neutral compound may have a pH in the range of 6 to 8. The pH of the compound may be measured by using a general pH measuring device, or may be measured by using a pH test paper. The pH measurement may be performed according to general measurement conditions.

Examples of the hydrophobic group include a long chain alkyl group, and a cyclic alkyl group. They may be used singly or in combination. At this time, if a fluorine atom is introduced to the long chain alkyl group or the cyclic alkyl group, a water repellent effect is made better.

The long chain alkyl group may be a straight chain type or a branched chain type. The number of carbon atoms of the long chain alkyl group is not particularly limited, but preferably 5 to 100 and more preferably 8 to 50. The cyclic alkyl group may be formed of a single cycle or plural cycles. The number of carbon atoms of the cyclic alkyl group is not particularly limited, but preferably 5 to 100 and more preferably 8 to 50. In the long chain alkyl group or the cyclic alkyl group, a carbon-carbon unsaturated bond, an amide bond, an ether bond, an ester bond or the like may be included.

The chelate group may be introduced by using various chelate ligands. Examples of the chelate ligands include β-dicarbonyl compound such as 1,3-diketone (β-diketone) and 3-keto carbonic acid ester (acetoacetic ester and the like), polyphosphate, aminocarbonic acid, hydroxycarbonic acid, polyamine, amino alcohol, aromatic heterocyclic bases, phenols, oximes, Schiff's base, tetrapyrroles, sulfur compound, synthesized macrocyclic compound, phosphonic acid, and hydroxyethylidenephosphonic acid. The compounds have plural unshared electron pairs capable of performing coordinate covalent bonding. They may be used singly or in combination. Among them, since 1,3-diketone and 3-keto carbonic acid ester do not have the acidic structure or base structure in the molecular structure and are neutral compounds, they are more preferable in terms of safety and preservation stability.

The specific examples of various chelate ligands include polyphosphates such as sodium tripolyphosphate and hexamethaphosphoric acid. Examples of the aminocarbonic acid include ethylenediamine diacetic acid, ethylenediamine dipropionic acid, ethylenediamine tetraacetic acid, N-hydroxymethylethylenediamine triacetic acid, N-hydroxyethylethylenediamine triacetic acid, diaminocyclohexyl tetraacetic acid, diethylenetriamine pentaacetic acid, glycoletherdiamine tetraacetic acid, N,N-bis(2-hydroxybenzyl)ethylenediamine diacetic acid, hexamethylenediamine N,N,N,N-tetraacetic acid, hydroxyethylimino diacetic acid, imino diacetic acid, diaminopropan tetraacetic acid, nitrilo triacetic acid, nitrilo tripropionic acid, triethylenetetramine hexaacetic acid, and poly(p-vinylbenzylimino diacetic acid).

Examples of 1,3-diketone include acetylacetone, trifluoroacetylacetone, and thenoyltrifluoroacetone. In addition, examples of acetoacetic ester include acetoacetic acid propyl, acetoacetic acid tent-butyl, acetoacetic acid isobutyl, and acetoacetic acid hydroxypropyl. Examples of hydroxycarbonic acid include N-dihydroxyethylglycine, ethylene bis(hydroxyphenylglycine), diaminopropanol tetraacetic acid, tartaric acid, citric acid, and gluconic acid. Examples of polyamine include ethylenediamine, triethylenetetramine, triaminotriethylamine, and polyethyleneimine. Examples of aminoalcohol include triethanolamine, N-hydroxyethylethylenediamine, and polymetharyloylacetone.

Examples of aromatic heterocyclic base include dipyridyl, o-phenanthroline, oxine, and 8-hydroxyquinoline. Examples of phenols, 5-sulfosalicylic acid, salicylaldehyde, disulfopyrocatecol, chronotropic acid, oxysulfonic acid, and disalicylaldehyde. Examples of oxime include dimethylglyoxime and salicylaldoxime. Examples of the Schiff's base include dimethylglyoxime, salicylaldoxime, disalicylaldehyde, and 1,2-propylenediamine.

Examples of tetrapyrroles include phthalocyanine and tetraphenylporpyrine. Examples of the sulfur compounds include toluenedithiol, dimercaptopropanol, thioglycol acid, potassium ethylxanthinate, sodium diethyldithiocarbamate, dithizone, and diethylthiophosphoric acid. Examples of the synthesized macrocyclic compounds include tetraphenylporpyrine and crown ethers. Examples of the phosphonic acids include ethylenediamine N,N-bismethylenephosphonic acid, ethylenediaminetetrakismethylenephosphonic acid, nitrilotrismethylenephosphonic acid, and hydroxyethylidenediphosphonic acid.

A hydroxyl group or an amino group may be appropriately introduced to the chelate ligand. Some of the chelate ligands are present in the form of salt. In this case, they may be used in the form of salt. In addition, a hydrate or solvated material of the chelate ligand or the chelate ligand in the form of the salt may be used. In addition, the chelate ligand, which includes an optical active structure may include a steric isomer, a mixture of steric isomers, or a racemic mixture.

The long chain alkyl group may be introduced by using the long chain alkyl compound. The long chain alkyl compound is not particularly limited, and examples thereof include long chain alkyl carbonic acid derivatives such as long chain alkyl carbonic acid, long chain alkyl carbonic acid ester, and long chain alkyl carbonic acid amide, long chain alkyl alcohol, long chain alkyl thiol, long chain alkyl aldehyde, long chain alkyl ether, long chain alkyl amine, long chain alkyl amine derivative, and long chain alkyl halogen. Among them, in terms of easy introduction of the chelate group, long chain alkyl carbonic acid, long chain alkyl carbonic acid derivative, long chain alkyl alcohol, and long chain alkyl amine are preferable.

Examples of the long chain alkyl compounds include octanic acid, nonaic acid, decanoic acid, hexadecanoic acid, octadecanoic acid, Icosanoic acid, docosanoic acid, tetradocosanoic acid, hexadocosanoic acid, octadocosanoic acid, octanol, nonanol, decanol, dodecanol, hexadecanol, octadecanol, eicosanol, docosanol, tetradocosanol, hexadocosanol, octadocosanol, octylamine, nonylamine, decylamine, dodecylamine, hexadecylamine, octadecylamine, dodecyl carbonic acid chloride, hexadecylcarbonic acid chloride, and octadecylcarbonic acid chloride. Among them, in terms of easy purchase, octanic acid, nonaic acid, decanoic acid, dodecanoic acid, ocutadecanoic acid, docosanoic acid, octanol, nonanol, decanol, dodecanol, octadecanol, docosanol, octylamine, nonylamine, decylamine, dodecylamine, octadecylamine, dodecyl carbonic acid chloride, and octadecylcarbonic acid chloride are preferable.

The cyclic alkyl group may be introduced by using the cyclic alkyl compound. The cyclic alkyl compound is not particularly limited, and examples thereof include a cyclo alkyl compound having 3 to 8 carbon atoms, a compound having a steroidal skeleton, and a compound having an adamantyl skeleton. At this time, in terms of easy formation of the bond to the chelate ligand, it is preferable that the carbonic acid group, the hydroxyl group, the acid amide group, the amino group, or the thiol group is introduced to the compounds described above.

Examples of the cyclic alkyl compound include cholic acid, deoxycholic acid, adamantane carbonic acid, adamantane acetic acid, cyclohexyl cyclohexanol, cyclopentadecanol, isoborneol, adamantanol, methyladamantanol, ethyladamantanol, cholesterol, cholestanol, cyclooctylamine, cyclododecylamine, adamantanemethylamine, and adamantaneethylamine. Among them, in terms of easy purchase, adamantanol and cholesterol are preferable.

Since the rust inhibitor according to the present invention has the hydrophobic group and the chelate group, the rust inhibitor may be obtained by contacting a compound having the hydrophobic group with the chelate ligand having the chelate group. To be specific, it may be obtained by performing condensation reaction between the compound having the hydrophobic group and the chelate ligand having the chelate group. At this time, a solvent may be used, and stirring may be performed. In addition, in order to increase a reaction rate, it may be heated or a catalyst may be added thereto. In addition, a target material may be obtained at high yield by removing a byproduct to make an equilibrium reaction biased toward a production system. Examples of the compound having the hydrophobic group include the long chain alkyl compound and the cyclic alkyl compound.

For example, when the compound having the hydrophobic group has a carboxyl group or hydroxyl group, and the chelate ligand has a hydroxyl group or carboxyl group, the hydrophobic group and the chelate group may be bonded to each other by the ester bond. In addition, for example, when the compound having the hydrophobic group has a carboxyl group or amino group, and the chelate ligand has an amino group or carboxyl group, the hydrophobic group and the chelate group may be bonded to each other by the amide bond.

The molecular weight of the compound that is an effective component of the rust inhibitor according to the present invention is not particularly limited, but preferably 100 to 1500 and more preferably 200 to 800.

An example of the compound that is an effective component of the rust inhibitor according to the preferred embodiment of the present invention is shown in the following Structural Formula.

[Formula 1]

R—X—Y (1)

where R is the long chain alkyl group or the cyclic alkyl group, X is an ester bond portion, an ether bond portion, a thioester bond portion, or an amide bond portion, and Y is a chelate group. That is, the long chain alkyl group or cyclic alkyl group and the chelate group are bonded to each other by the ester bond, ether bond, thioester bond, or amide bond.

The rust inhibitor according to the present invention may contain other components in addition to the compound that is the effective component. Examples of the additional components include an organic solvent, wax, and oil. The additional components may have the rust inhibiting effect, or may not have the rust inhibiting effect. The additional components have a function of a diluting agent. That is, according to the property and shape (liquid phase, solid, or powder) of the compound that is the effective component of the rust inhibitor according to the present invention, the additional components control the property and shape of the rust inhibitor in order to easily perform coating.

When the additional components are contained, it is preferable that the content of the effective component in the composition constituting the rust inhibitor is 0.01 mass % or more. More preferably, it is in the range of 0.05 to 99.5 mass %. If the content of the effective component is less than 0.01 mass %, the rust inhibiting effect is easily reduced. Examples of the organic solvent of the additional component include oxygen-containing solvents such as alcohols having 1 to 8 carbon atoms, tetrahydrofurane, and acetone, and alkanes having 6 to 18 carbon atoms. In addition, examples of the wax include polyethylene wax, synthetic paraffins, natural paraffins, microwax, and chlorinated hydrocarbons. In addition, examples of the oil include lubricant, operation oil, thermal medium oil, and silicon oil.

When the rust inhibitor according to the present invention is coated on the metal surface, the compound that is the effective component or a mixture of the compound and the additional components is directly coated on the metal surface. At this time, methods such as a coating method, a precipitation method, and a spray method may be used as the coating method. In addition, after coating treatment by a squeeze coater, precipitation treatment, or spray treatment, the coating amount may be controlled and an appearance and film thickness may be made uniform by an air knife method or a roll squeeze method. When the coating is performed, in order to improve an adhering property and a corrosion resistance, treatments such as heating or compression may be performed as needed.

Next, a surface treatment metal material according to another preferred embodiment of the present invention will be described. The surface treatment metal material according to the preferred embodiment of the present invention is obtained by coating the rust inhibitor according to the present invention on a surface of a metal material. It is preferable that the metal material is made of metal such as aluminum, iron, copper, an aluminum alloy, an iron alloy, and a copper alloy. At this time, the surface of the metal material may be plated with metal such as zinc or aluminum. The above-mentioned coating methods may be used as the coating method of the rust inhibitor.

The surface treatment metal material according to the preferred embodiment of the present invention may preferably be used for metal parts such as wires, cables, connectors, and bodies in vehicles such as automobiles, and metal parts such as high voltage power cables, electric and electronic device.

### Examples

Hereinafter, the present invention is described in detail by Examples, but the present invention is not limited to them.

(Experimental Material and Manufacturer)

The experimental materials used in the present Examples and Comparative Examples are described in conjunction with manufacturers and trade names. In addition, some of them were materials synthesized in the laboratory. With respect to the synthetic products, synthesis methods, structural formulae, and identification data are described below. In addition, the materials without the manufacturers and the trade names mean chemical reagents.

(A) Synthesis of the Compound that is the Effective Component of the Rust Inhibitor

Synthesis of Compound A (Compound Represented by Formula 2)

Five grams of ethylenediamine tetraacetic acid dianhydride (19.5 mmol) was dissolved in 50 ml of toluene, and 5.3 g of octadecyl alcohol (19.6 mmol) was dissolved. After the mixture solution was stirred at room temperature for 5 hours, the temperature was increased to 80° C. and additional stirring was performed for 1 hour. After the reaction was finished, while the reaction solution was cooled and stirred in an ice bath, 200 ml of pure water was added little by little. Thereafter, the temperature was cooled to room temperature and the stirring was performed for 1 hour, and the toluene phase was separated and concentrated in vacuo. Methanol and water were continuously added to the concentrated material, and the precipitate was obtained by filtration to obtain a light yellow powder. The powder was recrystallized in methanol, and filtered to obtain a light yellow target material (yield 65%). 1H-NMR(DMSO)σppm(TMS): 0.85 (t, 3H), 1.25 (m, 32H), 1.55 (t, 2H), 2.79 (m, 4H), 3.47 (m, 11H), 4.03 (t, 2H). IR(cm⁻¹): 2925 (C—H stretching), 1734 (ester C═O stretching), 1460 (carbonic acid C—O stretching), 1225 (ester C—O stretching), 1060 (C—N stretching).

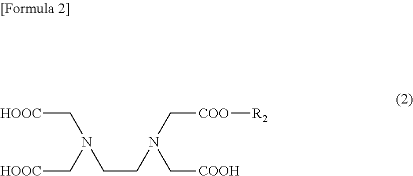

where R₂ is an octadecyl group.

Synthesis of Compound B (Compound Represented by Formula 3)

Five grams of diethylenediamine pentaacetic acid 2 anhydride (14.0 mmol) was dissolved in 50 ml of toluene, and 4.6 g of docosanol (14.0 mmol) was dissolved. After the mixture solution was stirred at room temperature for 5 hours, the temperature was increased to 80° C. and additional stirring was performed for 1 hour. After the reaction was finished, while the reaction solution was cooled and stirred in an ice bath, 200 ml of pure water was added little by little. Thereafter, the temperature was cooled to room temperature and the stirring was performed for hour, and the toluene phase was separated and concentrated in vacuo. Methanol and water were continuously added to the concentrated material, and the precipitate was obtained by filtration to obtain a light yellow powder. The powder was recrystallized in methanol, and filtered to obtain a light yellow target material (yield 56%). 1H-NMR(DMSO)σppm(TMS): 0.86 (t, 3H), 1.25 (m, 40H), 1.57 (t, 2H), 2.79 (m, 8H), 3.37 (s, 2H), 3.41 (m, 6H), 3.49 (s, 2H), 4.04 (t, 2H). IR(cm-1): 2910 (C—H stretching), 1734 (ester C═O stretching), 1455 (carbonic acid C—O stretching), 1225 (ester C—O stretching), 1070 (C—N stretching).

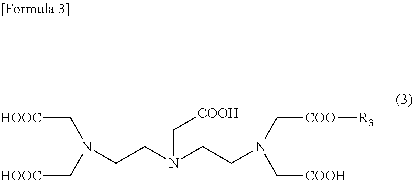

where R₃ is a dococyl group.

Synthesis of Compound C (Compound Represented by Formula 4)

Five grams of tert-butylacetoacetate (31.6 mmol) and 8.5 g of octadecylalcohol (31.4 mmol) were dissolved in 50 ml of toluene, and heated to 110° C. while they were stirred, and reacted for 2 hours while tert-butanol that was the byproduct was removed using a Dean-Stark trap. After the reaction was finished, it was concentrated in vacuo to obtain a white composition in a wax state. Twenty milliliters of cold water was added thereto to solidify it, and a target material was obtained by filtration (yield 75%). 1H-NMR(CDCl₃)σppm(TMS): 0.89 (t, 3H), 1.26 (m, 32H), 1.64 (m, 2H), 2.27 (s, 3H), 3.44 (s, 2H), 4.13 (t, 2H). IR(cm⁻¹): 2924 (C—H stretching), 1745, 1720 (β-diketone, enol form, 1642 (βdiketone, enol form), 1420 (carbonic acid C—O stretching).

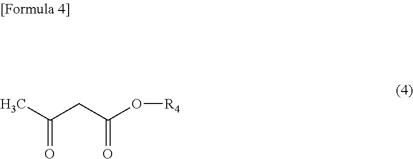

where R₄ is an octadecyl group.

Synthesis of Compound D (Compound Represented by Formula 5)

The compound was synthesized by using the same method as compound C, except that 10.3 g of docosanol (31.5 mmol) was used instead of octadecyl alcohol (yield 78%). 1H-NMR(CDCl₃)σppm(TMS): 0.89 (t, 3H), 1.27 (m, 40H), 1.64 (m, 2H), 2.25 (s, 3H), 3.44 (s, 2H), 4.10 (t, 2H). IR(cm⁻¹): 2922 (C—H stretching), 1745, 1721 (β-diketone, enol form), 1650 (β-diketone, enol form), 1425 (carbonic acid C—O stretching).

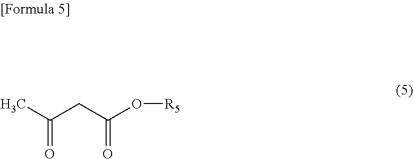

where R₅ is a dococyl group.

Synthesis of compound E (Compound Represented by Formula 6)

While 5 g of hydroxyethylimino diacetic acid (28.2 mmol) was dissolved in 200 ml of DMF, cooled and stirred in a water bath, 8.6 g of stearoyl chloride (28.4 mmol) was added little by little. After that, the stirring was continued at room temperature for 12 hours. After the reaction was finished, while the reaction solution was cooled and stirred in an ice bath, 200 ml of pure water was added little by little. After the temperature was cooled to room temperature and the stirring was performed for 1 hour, the pH was controlled to 2.0 using a 1N sodium hydroxide solution, and the mixture solution thereof was concentrated. Two hundred milliliters of pure water was added to the obtained brown oil, which was cleaned twice by decantation thereafter. The cleaned material was dissolved in methanol by heat, cooled, recrystallized, and filtered to obtain a light yellow powder. The recrystallization of methanol was repeated once more to obtain a light yellow target material (yield 67%). 1H-NMR(DMSO)σppm(TMS): 0.86 (t, 3H), 1.24 (m, 30H), 1.57 (t, 2H), 2.34 (t, 2H), 2.44 (t, 2H), 3.48 (m, 6H), 4.03 (t, 2H). IR(cm⁻¹): 2923 (C—H stretching), 1730 (ester C═O stretching), 1455 (carbonic acid C—O stretching), 1220 (ester C—O stretching), 1058 (C—N stretching).

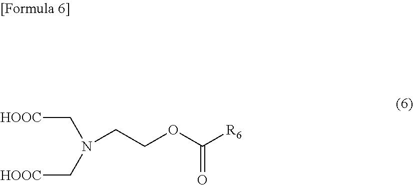

where R₆ is a heptadecyl group.

Synthesis of Compound F (Compound Represented by Formula 7)

The compound was synthesized by using the same method as compound E, except that 7.9 g of N-(2-hydroxyethyl)ethylenediamine triacetic acid (28.4 mmol) was used instead of hydroxyethylimino diacetic acid (yield 51%). 1H-NMR(DMSO)σppm(TMS): 0.87 (t, 3H), 1.24 (m, 30H), 1.57 (t, 2H), 2.37 (t, 2H), 2.48 (t, 2H), 3.45 (m, 9H), 4.02 (t, 2H). IR(cm⁻¹): 2925 (C—H stretching), 1733 (ester C═O stretching), 1453 (carbonic acid C—O stretching), 1220 (ester C—O stretching), 1060 (C—N stretching).

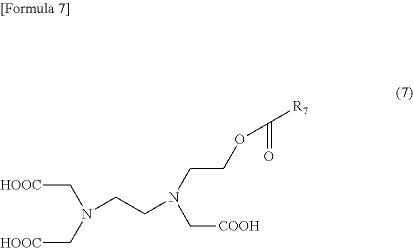

where R₇ is a heptadecyl group.

Synthesis of Compound G (Compound Represented by Formula 8)

The compound was synthesized by using the same method as compound E, except that 9.2 g of diaminopropanol tetraacetic acid (28.5 mmol) was used instead of hydroxyethylimino diacetic acid (yield 47%). 1H-NMR(DMSO)σppm(TMS): 0.85 (t, 3H), 1.24 (m, 30H), 1.56 (t, 2H), 2.56 (m, 2H), 2.75 (m, 2H), 3.45 (m, 8H), 3.87 (m), 1H), 4.02 (t, 2H). IR(cm⁻¹): 2922 (C—H stretching), 1735 (ester C═O stretching), 1453 (carbonic acid C—O stretching), 1220 (ester C—O stretching), 1060 (C—N stretching).

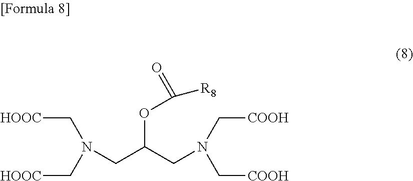

where R₈ is a heptadecyl group.

Synthesis of Compound H (Compound Represented by Formula 9)

The compound was synthesized by using the same method as compound E, except that 5.9 g of 1-hydroxyethane-1,1-bisphosphonic acid (28.6 mmol) was used instead of hydroxyethylimino diacetic acid (yield 54%). 1H-NMR(DMSO)σppm(TMS): 0.87 (t, 3H), 1.24 (m, 30H), 1.49 (s, 3H), 1.61 (t, 2H), 4.00 (t, 2H). IR(cm⁻¹): 2925 (C—H stretching), 1730 (ester C═O stretching), 1450 (C—O stretching), 1151 (P—O stretching), 925(P—OH).

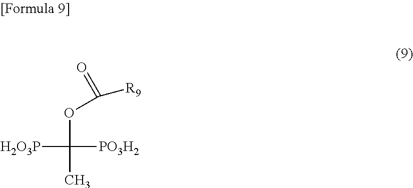

where R₉ is a heptadecyl group.

Synthesis of Compound I (Compound Represented by Formula 10)

The compound was synthesized by using the same method as compound A, except that 7.5 g of cholesterol (19.4 mmol) that had the structure represented by following Formula 14 was used instead of octadecylalcohol (yield 59%). 1H-NMR(DMSO)σppm(TMS): 0.5 to 2.0 (m, 41H), 2.28 (m, 2H), 3.47 (m, 11H), 3.52 (m, 12H), 5.35 (m, 1H). IR(cm⁻¹): 2925 (C—H stretching), 1734 (ester C═O stretching), 1460 (carbonic acid C—O stretching), 1225 (ester C—O stretching), 1060 (C—N stretching).

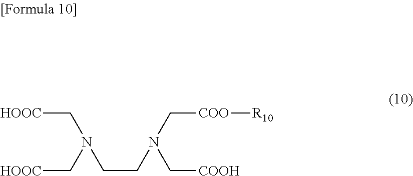

where R₁₀ is a cholesteryl group.

Synthesis of Compound J (Compound Represented by Formula 11)

The compound was synthesized by using the same method as compound B, except that 2.1 g of 1-adamantanol (13.8 mmol) that had the structure represented by following Formula 15 was used instead of docosanol (yield 48%). 1H-NMR(DMSO)σppm(TMS): 1.71 (m, 12H), 2.14 (m, 3H), 2.79 (m, 8H), 3.36 (s, 2H), 3.50 (m, 6H). IR(cm⁻¹): 2954, 2922 (C—H stretching), 1735 (ester C═O stretching), 1455 (carbonic acid C—O stretching), 1225 (ester C—O stretching), 1070 (C—N stretching).

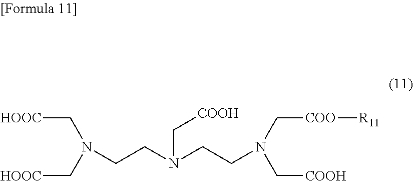

where R₁₁ is an adamantyl group.

Synthesis of Compound K (Compound Represented by Formula 12)

The compound was synthesized by using the same method as compound C, except that 12.1 g of cholesterol (31.3 mmol) that had the structure represented by following Formula 14 was used instead of octadecylalcohol (yield 48%). 1H-NMR(CDCl₃)σppm(TMS): 0.5 to 2.0 (m, 41H), 2.28 (m, 2H), 2.26 (s, 3H), 3.41 (s, 2H), 3.52 (m, 1H), 5.35 (m, 1H). IR(cm⁻¹): 2925 (C—H stretching), 1745, 1720 (β-diketone, enol form), 1642 (β-diketone, enol form), 1440 (carbonic acid C—O stretching).

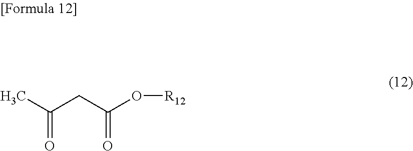

where R₁₂ is a cholesteryl group.

Synthesis of Compound L (Compound Represented by Formula 13)

The compound was synthesized by using the same method as compound C, except that 4.8 g of 1-adamantanol (31.5 mmol) that had the structure represented by following Formula 15 was used instead of octadecylalcohol (yield 48%). 1H-NMR(CDCl₃)σppm(TMS): 1.71 (m, 12H), 2.14 (m, 3H), 2.25 (s, 3H), 3.44 (s, 2H). IR(cm-1): 2930 (C—H stretching), 1745, 1722 (β-diketone, enol form), 1645 (β-diketone, enol form), 1444 (carbonic acid C—O stretching).

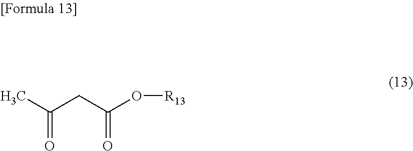

where R₁₃ is an adamantyl group.

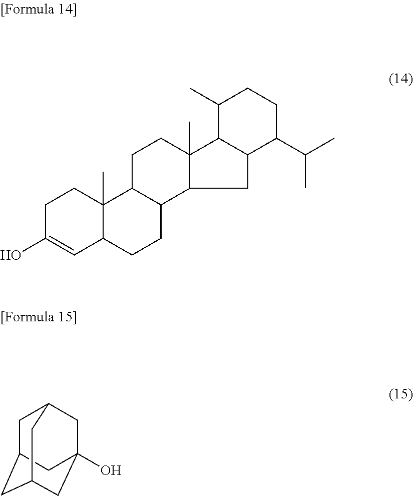

(B) Additional Components (Diluting Agent)

Wax <1> [trade name “LUVAX 1151”, manufactured by NIPPON SEIRO, CO., LTD.]

Wax <2> [trade name “CERIDUST 3620”, manufactured by HOECHST AG]

Oil [trade name “DAPHNE MECHANIC OIL 10”, manufactured by IDEMITSU KOSAN, CO., LTD.]

isopropyl alcohol (IPA) (reagent)

(Coating Method on the Metal Surface)

One milligram of compounds A to L that were synthesized by using the above-mentioned method was uniformly coated by providing the compounds on aluminum plates (10×10×0.5 mm) that were cleaned with ethanol, heating them at 100° C. for 5 minutes, and melting them to increase the fluidity. Thereafter, heating was stopped, and natural cooling was performed to room temperature to obtain each sample.

(Rust Inhibiting Test Method)

Ten microliters of 5% neutral saline solution was dropped on the side of each sample where the rust inhibitor was coated, the sample on which the 5% saline solution was spotted was subjected to a high temperature and high humidity test under the condition of 80° C., 95% RH, and 50 to 200 hours, the surface thereof was washed with pure water after a predetermined time, and the surface state of the portion of the sample that was spotted with the saline solution was observed and checked for generation of the white rust. At this time, the spotted surface was photographed, and the area ratio of white rust generation to the entire coated side of the rust inhibitor was obtained. The case where there was no white rust was classified as “Excellent”, the case where even though there was the white rust, the area ratio of the white rust generation was less than 5% was classified as “Good+”, the case where the area ratio of the white rust generation was 5% or more and less than 10% was classified as “Good”, the case where the area ratio of the white rust generation was 10% or more and less than 25% was classified as “Good−”, the case where the area ratio of the white rust generation was 25% or more and less than 50% was classified as “Below average”, and the case where the area ratio of the white rust generation was 50% or more was classified as “Not good”. The rust inhibiting test results are described in Table 1.

TABLE 1

| | Rust inhibitor | After 50 h | After 100 h | After 200 h |
|---|---|---|---|---|
| Example 1 | Compound A | Excellent | Excellent | Good+ |
| Example 2 | Compound B | Excellent | Excellent | Excellent |
| Example 3 | Compound C | Excellent | Excellent | Excellent |
| Example 4 | Compound D | Excellent | Excellent | Excellent |
| Example 5 | Compound E | Excellent | Excellent | Good+ |
| Example 6 | Compound F | Excellent | Excellent | Excellent |
| Example 7 | Compound G | Excellent | Excellent | Excellent |
| Example 8 | Compound H | Excellent | Excellent | Excellent |
| Example 9 | Compound I | Excellent | Excellent | Good+ |
| Example 10 | Compound J | Excellent | Excellent | Excellent |
| Example 11 | Compound K | Excellent | Excellent | Excellent |
| Example 12 | Compound L | Excellent | Excellent | Excellent |
| Comparative | Wax <1> | Good+ | Good | Not good |
| Example 1 | | | | |
| Comparative | Wax <2> | Good− | Good− | Below |
| Example 2 | | | | average |
| Comparative | None | Below | Not good | Not good |
| Example 3 | | average | | |

According to Table 1, with the commercial wax coat, under the high temperature and high humidity condition, the rust inhibiting effect was reduced by a contact to the saline solution over a long period of time to generate the rust. However, when the rust inhibitor according to the preferred embodiment of the present invention was used, it was confirmed that because of the strong bond to the aluminum surface of the chelate portion, the rust inhibiting effect was continuously obtained over a long period of time.

Subsequently, rust inhibitor compositions including respective compounds A to L were prepared by using the diluting agent that will be described in Table 2, and the rust inhibiting test was performed by using the compositions. The test was performed in the same manner as the coating method on the metal surface and the rust inhibiting test method described above. The contents of compounds A to L are expressed in mass % in Table 2. Meanwhile, in coating the rust inhibitor composition, considering the specific gravity of the composition in the solution state, the rust inhibitor composition was provided on the aluminum plate so that the amount thereof was 1 mg in a liquid state, and uniformly coated at 100° C. for 5 minutes. In addition, with respect to a case where the diluting agent was a volatile solvent, the rust inhibiting effect was evaluated by vaporizing only the diluting agent at 100° C. for 5 minutes after it was verified that diluting agent was sufficiently uniformly spread before volatilization. The results are shown in Table 2.

TABLE 2

| | Rust inhibitor | | | | | |
|---|---|---|---|---|---|---|
| | | | Diluting | After | | |
| | Compund | Content | agent | 50 h | After 100 h | After 200 h |
| Example 13 | A | 50 | Wax <1> | Excellent | Excellent | Good+ |
| Example 14 | B | 50 | Wax <1> | Excellent | Excellent | Good+ |
| Example 15 | C | 50 | Wax <1> | Excellent | Excellent | Excellent |
| Example 16 | D | 50 | Wax <1> | Excellent | Excellent | Excellent |
| Example 17 | E | 50 | Wax <1> | Excellent | Good+ | Good+ |
| Example 18 | F | 50 | Wax <1> | Excellent | Excellent | Excellent |
| Example 19 | G | 50 | Wax <1> | Excellent | Excellent | Excellent |
| Example 20 | H | 50 | Wax <1> | Excellent | Excellent | Excellent |
| Example 21 | I | 50 | Wax <1> | Excellent | Good+ | Good+ |
| Example 22 | J | 50 | Wax <1> | Excellent | Excellent | Excellent |
| Example 23 | K | 50 | Wax <1> | Excellent | Excellent | Excellent |
| Example 24 | L | 50 | Wax <1> | Excellent | Excellent | Excellent |
| Example 25 | C | 50 | Oil | Excellent | Excellent | Excellent |
| Example 26 | C | 50 | IPA | Excellent | Excellent | Excellent |
| Example 27 | D | 50 | Oil | Excellent | Good+ | Good+ |
| Example 28 | D | 50 | IPA | Excellent | Excellent | Good+ |
| Example 29 | K | 50 | Oil | Excellent | Excellent | Excellent |
| Example 30 | K | 50 | IPA | Excellent | Excellent | Excellent |
| Example 31 | C | 10 | IPA | Excellent | Excellent | Excellent |
| Example 32 | C | 5 | IPA | Excellent | Excellent | Excellent |
| Example 33 | C | 1 | IPA | Excellent | Excellent | Excellent |
| Example 34 | C | 0.5 | IPA | Excellent | Excellent | Excellent |
| Example 35 | C | 0.1 | IPA | Excellent | Excellent | Good+ |
| Example 36 | C | 0.05 | IPA | Excellent | Good+ | Good+ |
| Example 37 | K | 1 | IPA | Excellent | Excellent | Excellent |
| Example 38 | K | 0.5 | IPA | Excellent | Excellent | Excellent |
| Example 39 | K | 0.1 | IPA | Excellent | Excellent | Excellent |
| Example 40 | K | 0.05 | IPA | Excellent | Good+ | Good+ |
| Example 41 | C | 1 | Wax <1> | Excellent | Excellent | Excellent |
| Example 42 | C | 0.1 | Wax <1> | Excellent | Excellent | Good+ |

According to Table 2, it was confirmed that even if the rust inhibitor according to the present invention was diluted by the commercial wax or oil or the organic solvent, the rust inhibiting effect was shown over a long period of time, and the rust inhibiting effect was maintained even at the low concentration where the content was 0.05%.

Next, pH measurement was performed with respect to the rust inhibitors and the chelating agents described in Table 3. Among the compounds described in Table 3, compounds C, D, K, L, G, and H were the same compounds as those described in Tables 1 and 2, and compounds M and N were synthesized by using the following method. In addition, compounds O to R were the commercial reagents. Compounds C, D, K, L, M, G, H, and N were the compounds having the hydrophobic group and the chelate group. Compound O was a representative polyamine chelating agent, compound P was a representative carbonic acid chelating agent, compound Q was a representative phosphoric acid chelating agent, and compound R was a representative amine chelating agent.

Synthesis of Compound M (Compound Represented by Formula 16)

The compound was synthesized by using the same method as compound E, except that 9.0 g of nonadecanoic acid chloride (28.4 mmol) was used instead of stearoyl chloride (yield 70%). 1H-NMR(DMSO)σppm(TMS): 0.86 (t, 3H), 1.25 (m, 32H), 1.58 (t, 2H), 2.34 (t, 2H), 2.44 (t, 2H), 3.48 (m, 6H), 4.03 (t, 2H). IR(cm-1): 2923 (C—H stretching), 1733 (ester C═O stretching), 1455 (carbonic acid C—O stretching), 1220 (ester C—O stretching), 1056 (C—N stretching).

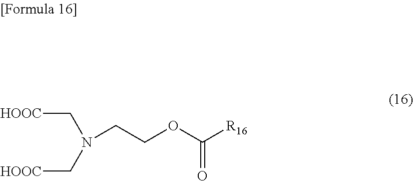

where R₁₆ is an octadecyl group.

Synthesis of Compound N (Compound Represented by Formula 17)

While 4.1 g of triethylenetetramine (28.0 mmol) was dissolved in 200 ml of DMF, cooled and stirred in a water bath, 8.6 g of stearoyl chloride (28.4 mmol) was added little by little. After that, the stirring was continued at room temperature for 12 hours. After the reaction was finished, while the reaction solution was cooled and stirred in an ice bath, 500 ml of pure water was added little by little. After the temperature was cooled to room temperature and the stirring was performed for 1 hour, while the 1N sodium hydroxide solution was added little by little, brown oil was formed at the pH of 11.0. The supernatant was removed, pure water was added to the obtained oil, which was cleaned twice by decantation thereafter. The cleaned material was dissolved in methanol by heat, cooled, recrystallized, and filtered to obtain a yellow powder. The recrystallization of methanol was repeated once more to obtain a light yellow target material (yield 58%). 1H-NMR(DMSO)σppm(TMS): 0.85 (t, 3H), 1.30 (m, 30H), 1.39 (t, 2H), 2.28˜2.81 (m, 12H), 3.60 (m, 5H). IR(cm-1): 3405 (N—H stretching), 2920 (C—H stretching), 1662 (amide C═O stretching), 1590 (N—H variable angle), 1050 (C—N stretching).

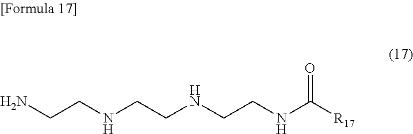

where R₁₇ is a heptadecyl group.

compound O: Polyethyleneimine

compound P: ethylenediamine tetraacetic acid

compound Q: polyphosphoric acid

compound R: diethylenetriamine

(pH Measurement Method)

One possible case where effects of corrosion, etc. are considered when the rust inhibitor is attached to a portion that is not included in an intended coated side is a case where the rust inhibitor is attached to an organic material or skin. The surface state thereof may be fat soluble or water soluble. In addition, a case where the organic material or skin becomes wet by water or oil components may be considered. Therefore, with the assumption of the surface state including both the states, a filtering paper that was wetted by the mixture solution where isopropyl alcohol:pure water=1:1 was prepared, 0.5 mg of each of the compounds described in Table 3 was provided on the surface thereof and left at room temperature for 1 minute, and the pH at the contact surface between the compound and the filtering paper was measured. At this time, an universal pH test paper (length 5 cm, width 7 mm, manufactured by ADVANTEC) was used as the filtering paper, and the pH value was obtained based on a change in color at the contact surface. That is, the pH value was obtained by comparing to the standard color. The results are described in Table 3.

TABLE 3

| Rust inhibitor or | |
|---|---|
| chelating agent | pH |
| Compound C | 7 |
| Compound D | 7 |
| Compound K | 6 to 7 |
| Compound L | 6 to 7 |
| Compound M | 2 to 3 |
| Compound G | 2 to 3 |
| Compound H | 1 to 2 |
| Compound N | 11 |
| Compound O | 11 |
| Compound P | 3 |
| Compound Q | 2 |
| Compound R | 10 to 11 |

According to Table 3, compounds M, G, H, N, and O to R have the acid structure or base structure in the molecular structure thereof. Accordingly, as the pH measurement result, it showed an acidic or alkali property. On the other hand, compounds C, D, K, and L are the neutral compounds that do not have the acid structure or base structure in the molecular structure thereof. Accordingly, the pH was neutral. Therefore, even when a rust inhibitor containing these compounds is used and the rust inhibitor is attached to a portion that is not included in an intended coated side, it is deemed that corrosion or effects to a human body are prevented. In addition, it is deemed that the preservation stability is excellent.

The foregoing description of the preferred embodiments of the present invention has been presented for purposes of illustration and description. However, it is not intended to limit the present invention to the preferred embodiments described herein, and modifications and variations are possible as long as they do not deviate from the principles of the invention.

1. A rust inhibitor comprising a compound that has a hydrophobic group and a chelate group in a molecular structure.

2. The rust inhibitor according to claim 1, wherein the hydrophobic group is one or a plurality of groups selected from the group consisting of a long chain alkyl group and a cyclic alkyl group.

3. The rust inhibitor according to claim 2, wherein the chelate group is derived from one or a plurality of chelate ligands selected from the group consisting of polyphosphate, aminocarbonic acid, 1,3-diketone, acetoacetic acid, acetoacetic ester, hydroxycarbonic acid, polyamine, amino alcohol, aromatic heterocyclic bases, phenols, oximes, Schiff's base, tetrapyrroles, sulfur compound, synthesized macrocyclic compound, phosphonic acid, and hydroxyethylidenephosphonic acid.

4. The rust inhibitor according to claim 3, wherein the hydrophobic group and the chelate group are bonded by one or a plurality of bonds selected from the group consisting of an ester bond, an ether bond, a thioester bond, a thioether bond, and an amide bond.

5. The rust inhibitor according to claim 4, wherein the non-polymerizable compound comprises a neutral compound.

6-8. (canceled)

9. The rust inhibitor according to claim 3, wherein the nonpolymerizable compound comprises a neutral compound.

10. The rust inhibitor according to claim 2, wherein the hydrophobic group and the chelate group are bonded by one or a plurality of bonds selected from the group consisting of an ester bond, an ether bond, a thioester bond, a thioether bond, and an amide bond.

11. The rust inhibitor according to claim 10, wherein the nonpolymerizable compound comprises a neutral compound.

12. The rust inhibitor according to claim 2, wherein the nonpolymerizable compound comprises a neutral compound.

13. The rust inhibitor according to claim 1, wherein the chelate group is derived from one or a plurality of chelate ligands selected from the group consisting of polyphosphate, aminocarbonic acid, 1,3-diketone, acetoacetic acid, acetoacetic ester, hydroxycarbonic acid, polyamine, amino alcohol, aromatic heterocyclic bases, phenols, oximes, Schiff's base, tetrapyrroles, sulfur compound, synthesized macrocyclic compound, phosphonic acid, and hydroxyethylidenephosphonic acid.

14. The rust inhibitor according to claim 13, wherein the hydrophobic group and the chelate group are bonded by one or a plurality of bonds selected from the group consisting of an ester bond, an ether bond, a thioester bond, a thioether bond, and an amide bond.

15. The rust inhibitor according to claim 14, wherein the nonpolymerizable compound comprises a neutral compound.

16. The rust inhibitor according to claim 13, wherein the nonpolymerizable compound comprises a neutral compound.

17. The rust inhibitor according to claim 1, wherein the hydrophobic group and the chelate group are bonded by one or a plurality of bonds selected from the group consisting of an ester bond, an ether bond, a thioester bond, a thioether bond, and an amide bond.

18. The rust inhibitor according to claim 17, wherein the nonpolymerizable compound comprises a neutral compound.

19. The rust inhibitor according to claim 1, wherein the nonpolymerizable compound comprises a neutral compound.

20. The rust inhibitor according to claim 1, wherein the rust inhibitor is used for metal surface coating.

21. A surface treatment metal material that is formed by coating the rust inhibitor according to claim 1, on a surface of a metal material.

22. The surface treatment metal material according to claim 21, wherein the metal material is made of one or a plurality of metals selected from the group consisting of aluminum, iron, copper, an aluminum alloy, an iron alloy, and a copper alloy.
